(19) United States
(12) Patent Application Publication
(10) Pub. No.: US 2026/0273265 A1
(43) Pub. Date: Sep. 17, 2026

(54) GUIDEWIRE FOR CIRCULATORY ASSIST DEVICES

(71) Applicant: Boston Scientific Scimed, Inc., Maple Grove, MN (US)

(72) Inventors: Tim O'Connor, Galway (IE); Chris Cullen, Galway (IE); Neil O'Connor, Galway (IE); Richard O'Sullivan, Galway (IE)

(73) Assignee: Boston Scientific Scimed, Inc., Maple Grove, MN (US)

(21) Appl. No.: 19/561,982

(22) Filed: Mar. 10, 2026

Related U.S. Application Data
(60) Provisional application No. 63/769,954, filed on Mar. 11, 2025.

(51) Int. Cl. A61M 60/865; A61M 60/13; A61M 60/216; A61M 60/411; A61M 60/81; A61M 60/90

(52) U.S. Cl. CPC A61M 60/865; A61M 60/13; A61M 60/216; A61M 60/411; A61M 60/81; A61M 60/90; A61M 2205/103; A61M 2210/12

(57) ABSTRACT

A percutaneous circulatory support system, including a guidewire and a blood pump advanceable along the guidewire within an introducer sheath. The guidewire includes a distal section having a first diameter, a central section having a second diameter, and a proximal section having a third diameter. The second diameter being greater than the first and third diameters. The blood pump includes an impeller housing, a motor housing coupled to the impeller housing, and an impeller disposed within the impeller housing. The impeller is configured to cause blood to flow through the blood pump. The blood pump is advanceable over the proximal section of the guidewire while passing through the introducer sheath.

### CROSS REFERENCE TO RELATED APPLICATIONS

This application claims the benefit of U.S. Provisional Patent Application Ser. No. 63/769,954 filed on Mar. 11, 2025, the disclosure of which is incorporated herein by reference.

### TECHNICAL FIELD

The disclosure pertains to percutaneous circulatory support systems. More specifically, the disclosure is related to percutaneous circulatory support devices and guidewires for positioning such devices within the heart

### BACKGROUND

A wide variety of medical devices have been developed for medical use including, for example, medical devices utilized to provide transient support in patients with compromised heart function or cardiac output. Generally, such devices, which may be referred to as percutaneous circulatory support devices or percutaneous blood pumps, are delivered to a patient's heart using a guidewire. However, there is an ongoing need to provide alternative configurations of guidewires for use with percutaneous blood pumps, and methods of advancement of the percutaneous blood pump along a guidewire into a patient's vasculature.

### SUMMARY

This disclosure provides design, material, manufacturing method, and use alternatives for medical devices, such as guidewires for use with percutaneous blood pumps.

An example is a percutaneous circulatory support system. The system includes a guidewire comprising a distal section having a first diameter, a central section having a second diameter, and a proximal section having a third diameter. The second diameter is greater than both the first diameter and the third diameter. The system also includes a blood pump configured to be coupled to and moved along the guidewire. The blood pump includes an impeller housing, a motor housing coupled to the impeller housing, and an impeller disposed within the impeller housing. The impeller is configured to cause blood to flow through the blood pump.

Alternatively or additionally to the embodiment above, the first and third diameters are equal.

Alternatively or additionally to the embodiments above, the first diameter is less than the third diameter.

Alternatively or additionally to the embodiments above, the guidewire further comprises a first transition section between the proximal section and the central section, and a second transition section between the central section and distal section.

Alternatively or additionally to the embodiments above, the percutaneous circulatory support device further includes an introducer sheath configured to receive the blood pump and the proximal section of the guidewire therethrough.

Alternatively or additionally to the embodiments above, the impeller housing comprises an inlet and an outlet, wherein the guidewire is configured to extend longitudinally through the impeller housing, proximally out of the outlet, and distally out of the inlet.

Alternatively or additionally to the embodiments above, the proximal section of the guidewire has a length greater than a length of the introducer sheath.

Alternatively or additionally to the embodiments above, the guidewire has an overall length of 180 cm (70.87 inches) to 250 cm (98.43 inches).

Alternatively or additionally to the embodiments above, the central section of the guidewire has a length between 300 mm (11.82 inches) and 330 mm (12.99 inches).

Alternatively or additionally to the embodiments above, the proximal section of the guidewire has a length between 140 cm (55.12 inches) and 210 cm (82.68 inches).

Alternatively or additionally to the embodiments above, the distal section of the guidewire has a length between 30 mm (1.18 inches) and 70 mm (2.76 inches). distal section of the guidewire comprises a tapered core, tapering to a smaller diameter toward a distal end of the guidewire.

Alternatively or additionally to the embodiments above, the first diameter is between 0.457 mm (0.018 inches) and 0.889 mm (0.035 inches).

Alternatively or additionally to the embodiments above, the second diameter is between 0.635 mm (0.025 inches) and 0.889 mm (0.035 inches).

Alternatively or additionally to the embodiments above, the third diameter is 0.457 mm (0.018 inches).

Another example is percutaneous circulatory support system. The system includes a guidewire including a distal section extending to a distal end of the guidewire, a central section, and a proximal section extending to a proximal end of the guidewire. The distal section has a first diameter, the central section has a second diameter, and the proximal section has a third diameter. The second diameter is greater than both the first diameter and the third diameter. The guidewire includes a first transition section between the proximal section and the central section and a second transition section between the central section and the distal section. The system also includes a blood pump configured to be coupled to and moved along the guidewire. The blood pump includes an impeller housing, a motor housing coupled to the impeller housing, and an impeller disposed within the impeller housing. The impeller is configured to cause blood to flow through the blood pump. The system also includes an introducer sheath configured to receive the blood pump and the proximal section of the guidewire therethrough.

Alternatively or additionally to the embodiments above, the first and third diameters are equal.

Alternatively or additionally to the embodiments above, the first and third diameters are between 0.457 mm (0.018 inches) and 0.635 mm (0.025 inches).

Alternatively or additionally to the embodiments above, the second diameter is between 0.635 mm (0.025 inches) and 0.889 mm (0.035 inches).

Another example is a method of positioning a blood pump within a patient. The method includes inserting an introducer sheath at an access point, and inserting a guidewire into the introducer sheath. The guidewire has a distal section with a first diameter, a central section with a second diameter, and a proximal section with a third diameter. The first and third diameters are less than the second diameter. The method further includes positioning the guidewire through the introducer sheath such that a distal end of the proximal section is distal of the introducer sheath, and thereafter, advancing the blood pump through the introducer sheath along the proximal section of the guidewire. The blood pump includes an impeller housing and an impeller disposed within the housing and configured to cause blood to flow through the blood pump.

Alternatively or additionally to the embodiments above, the third diameter is 0.635 mm (0.025 inches) or less and the second diameter is greater than 0.635 mm (0.025 inches).

Alternatively or additionally to the embodiments above, further including advancing the blood pump distally out of the introducer sheath and along the central section of the guidewire.

The above summary of some embodiments is not intended to describe each embodiment or every implementation of the present disclosure. The Figures and the Detailed Description, which follow, more particularly exemplify some of these embodiments.

### BRIEF DESCRIPTION OF THE DRAWINGS

The disclosure may be more completely understood in consideration of the following detailed description of various embodiments in connection with the accompanying drawings, in which:

FIG. 1 is a perspective view of an exemplary catheter including a percutaneous blood pump;

FIG. 2 shows a distal region of the catheter of FIG. 1 including the percutaneous blood pump;

FIG. 3 illustrates an example introducer sheath;

FIG. 4 illustrates inserting the percutaneous blood pump of FIGS. 1-2 through the introducer sheath of FIG. 3;

FIG. 5 illustrates an exemplary guidewire for use with a percutaneous blood pump;

FIG. 6A-6B illustrate tracking the percutaneous blood pump of FIGS. 1-2 over the guidewire of FIG. 5;

FIG. 7 illustrates an exemplary guidewire for use with a percutaneous blood pump, including a coiled distal end; and

FIG. 8 illustrates an exemplary guidewire for use with a percutaneous blood pump, including a floppy distal end.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about,” in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream,” “downstream,” “inflow,” and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum,” which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment,” “some embodiments,” “other embodiments,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Patients with compromised heart function or cardiac output may be treated with a percutaneous circulatory support device (e.g., a percutaneous blood pump). Such devices are often advanced through vasculature to reach a desired treatment area, such as a chamber of the heart (e.g., the left ventricle). In certain cases, it may be advantageous to use a large diameter guidewire when passing through tortuous anatomy. A smaller diameter guidewire may not provide enough rail support for tracking and may buckle when traversing tortuous vasculature having a tight vascular radii. However, a large diameter guidewire takes up already limited space within the lumen of an introducer sheath. Additionally, a large diameter guidewire may catch or bind to the percutaneous circulatory support device as the guidewire exits one of the outflow windows of the blood pump, reducing the efficiency of the procedure.

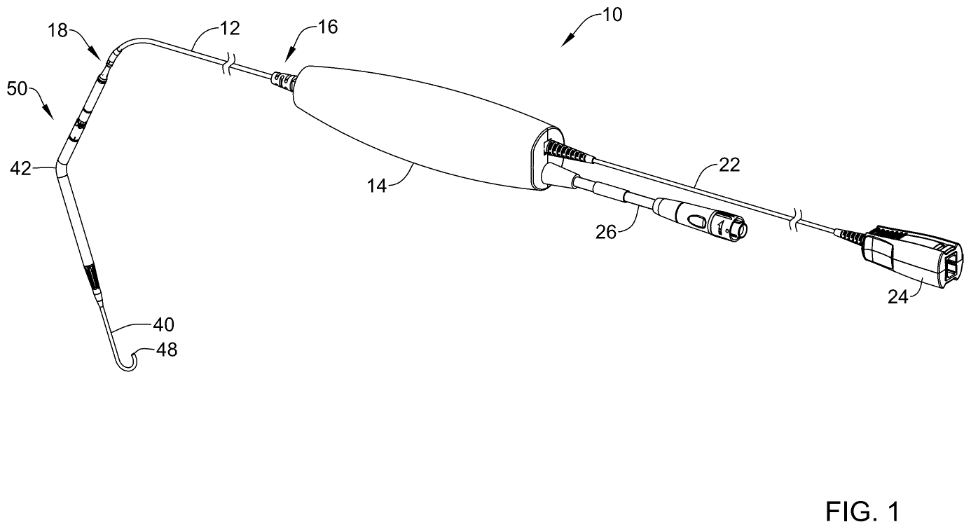

FIG. 1 illustrates a perspective view of a catheter 10 including a percutaneous blood pump 50 located at a distal end region thereof. The catheter 10 may be coupled to or include the blood pump 50, with an elongate shaft 12 of the catheter 10 extending proximally from the percutaneous blood pump 50 and a distal tip 40 extending distally from the blood pump 50. The distal tip 40 has and extends to a distal end 48. A distal portion of the blood pump 50 may be flexible, with a proximal portion being rigid. A bend 42 between the flexible and rigid portions of the blood pump 50 creates an inner curvature. A proximal end 16 of the elongate shaft 12 may be coupled to a junction housing 14 and a distal end 18 of the elongate shaft 12 may be coupled to the percutaneous blood pump 50. An electrical cable 22 may extend from the junction housing 14 to a connector 24 at a proximal end thereof. The connector 24 may be configured to be connected to a controller (not shown) for controlling the blood pump 50, such as providing electrical power to the blood pump 50. The catheter 10 may also include an extension 26 connectable to the controller for sending and/or receiving signals, such as from one or more sensors during operation of the blood pump 50.

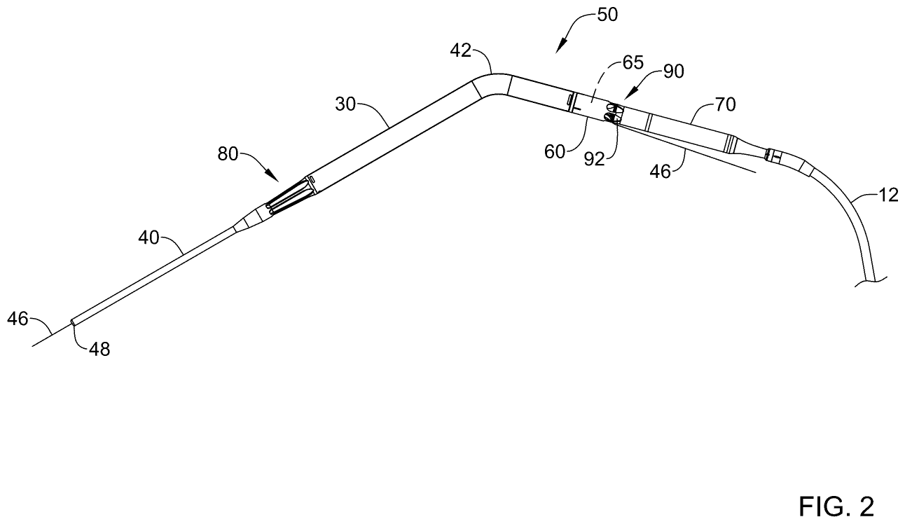

Additional features of the blood pump 50 are illustrated in FIG. 2. The blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 60 and/or the motor housing 70 may be separate components. The impeller housing 60 carries an impeller assembly 65 therein. The impeller assembly 65 may include an impeller secured to an impeller shaft that rotates relative to the impeller housing 60 to drive blood through the blood pump 50. In some embodiments, the impeller shaft and the impeller of the impeller assembly 65 may be integrally formed, whereas, in other embodiments the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows 92 arranged around a circumference of the impeller housing 60. In other embodiments, the inlet 80 and/or the outlet 90 may be formed on other portions of the blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller shaft may be directly or indirectly coupled to the drive shaft of the motor. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly 65. More specifically, in embodiments incorporating an impeller shaft, the impeller shaft and the impeller of the impeller assembly 65 are configured to rotate with the driven magnet. In other embodiments, the motor may be coupled to the impeller assembly 65 via other components.

Additionally, shown in FIG. 2, the catheter 10 is loaded onto a guidewire 46 for introduction of the blood pump 50 into a vasculature. In some embodiments the device may include a guidewire loading aid (not shown) through which a guidewire 46 may be routed when loading the catheter onto the guidewire. A guidewire 46, inserted through a guidewire lumen of the distal tip 40 (i.e., into the distal tip 40 through an opening at the distal end 48), may be advanced proximally along the impeller assembly 65 and out through one of the outflow windows 92 of the blood outlet 90, leaving the guidewire 46 tracked through the blood pump 50 such that the catheter 10 may be advanced over the guidewire 46 into a vasculature. The guidewire 46 may extend along the exterior of the elongate shaft 12 proximal of the blood pump 50 to a location external of the patient's body. Thus, the guidewire 46 may extend through the blood pump 50 from a distal opening at the distal end 48 of the distal tip 40 to one of the outflow windows 92 of the blood outlet 90, and then proximally therefrom along the exterior of the elongate shaft 12 to a proximal end of the catheter 10.

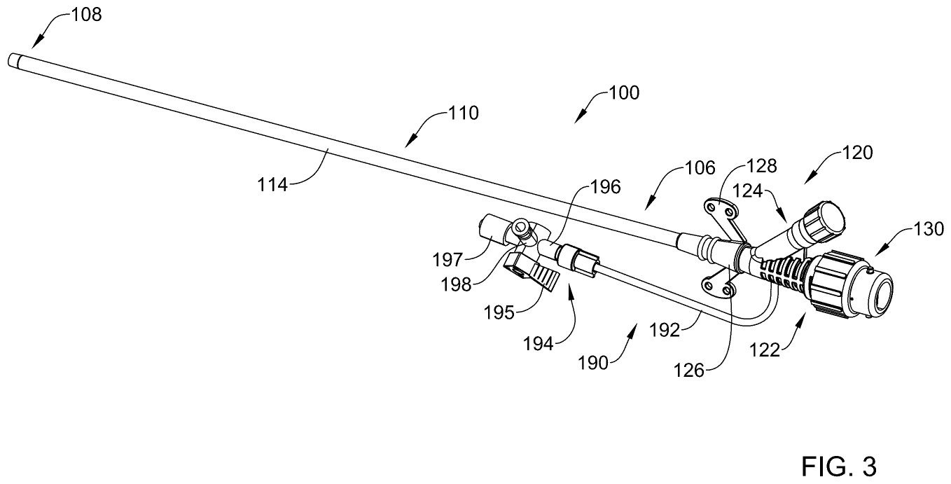

With reference to FIG. 3, an introducer sheath 100 may be provided for use in providing vascular access for introducing the percutaneous blood pump 50 of FIG. 1 into a vasculature at a vascular access site, such as a femoral access site. Generally, the introducer sheath 100 includes an elongate shaft 114 having a lumen with an inner diameter. The introducer sheath 100 may be constructed of various appropriate materials such as silicone, polyethylene, polyamide, or other medical grade synthetics or plastics. The elongate shaft 114 may include one or more components, which may include metals such as stainless-steel and nitinol to provide the introducer sheath 100 with structural integrity, flexibility, expansion/collapse properties or the like. In some embodiments, the elongate shaft 114 of the introducer sheath 100 may have an inner diameter in a range of 16 French to 24 French (0.210 inches to 0.315 inches), or about 16 French (e.g., about 0.209 to about 0.215 inches), for example. The outer diameter of the elongate shaft 114 may be in the range of about 0.225 inches to about 0.245 inches, or about 0.235 inches, for example. It should be understood that the elongate shaft 114 may be of any size and shape to accommodate a variety of blood pumps.

The introducer sheath 100 may include a hub 120, as well as the elongate shaft 114 extending distally from the hub 120 to a distal end region 108 and defining a body portion 110 of the sheath 100. The sheath 100 may also include a flush line 190 extending from the hub 120. The flush line 190 may include a tubular member 192 extending from the hub 120 and in fluid communication with a lumen of the hub 120. The tubular member 192 may extend to a stopcock 194, such as a three-way stopcock. The stopcock 194 may include a first leg 196 coupled to the tubular member 192, a second leg 197, a third leg 198, and a lever 195 that is rotatable to selectively open/close fluid access between the first, second and/or third legs 196, 197, 198. Each of the legs 196, 197, 198 may include a connector, such as a luer connector, as desired.

The hub 120 may also include a strain relief 126 configured to provide a transition in flexibility along a proximal end region 106 of the elongate shaft 114. The strain relief 126 may include a body attached to a main body of the hub 120, as will be described further herein. The strain relief 126 may include one or more suture pads 128 extending outward therefrom. For example, the strain relief 126 may include first and second suture pads 128 extending from opposite sides of the strain relief 126. The suture pads 128 may facilitate securing the hub 120 against the patient once the introducer sheath 100 has been positioned in the blood vessel of the patient. For example, each suture pad 128 may include at least one opening extending therethrough for receiving a suture used to suture the hub 120 to the skin of the patient.

The hub 120 may include a main port 122 and a side port 124 extending from the main port 122. In some instances, the side port 124 may extend at an acute angle from the main port 122. The main port 122 and/or the side port 124 may provide access to one or more lumens extending through the body portion 110 (e.g., through the elongate shaft 114) of the sheath 100. In some instances, the main port 122 may be a tightening port 130.

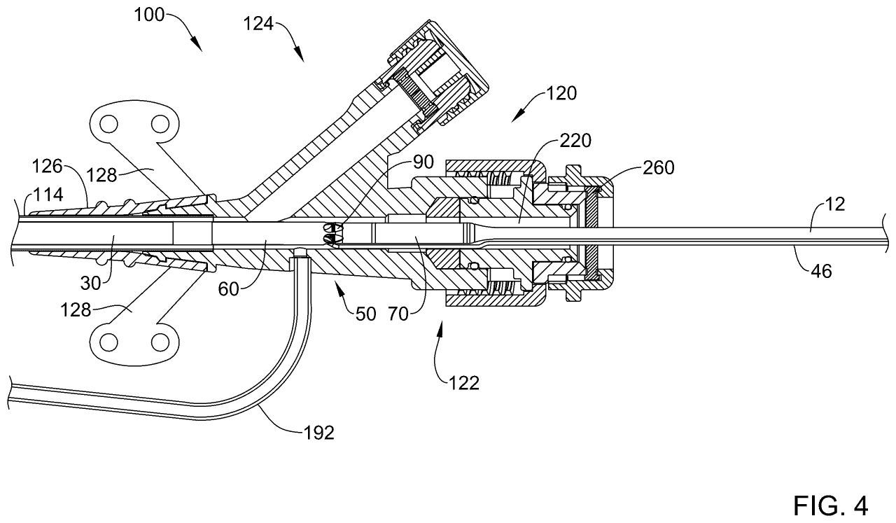

FIG. 4 illustrates the process of inserting the catheter 10, including the blood pump 50 on a distal end thereof, into a vasculature of a patient through the introducer sheath 100. The components of the catheter 10, including the blood pump 50, are shown in plain view while components of the introducer sheath 100 are shown in cross-section. The blood pump 50 (see also FIG. 1), may be advanced through an elastomeric seal 260, through the lumen of the hub 120 and into a passageway 220 of the main port 122, then into the lumen of the elongate shaft 114 of the introducer sheath 100. The elastomeric seal 260 may seal around an outer perimeter of the cannula 30 of the blood pump 50 to prevent blood from escaping from the main port 122. Once within the introducer sheath 100, the elongate shaft 12 of the catheter 10 may be further advanced through the introducer sheath 100 into the body of the patient to position the blood pump 50, including the cannula 30, the impeller housing 60, the motor housing 70, and the outflow windows 92, in a desired location such as across the aortic valve between the left ventricle of the heart and the aorta.

When within the introducer sheath 100, the space between the blood pump 50 and the interior wall of the elongate shaft 114 of the introducer sheath 100 is limited. A large diameter guidewire 46 can make it difficult to advance the blood pump 50 when within the introducer sheath 100. For example, a guidewire with a large diameter may bind to the outflow window 92 of the impeller housing 60 as the blood pump 50 is tracked along the guidewire 46. Furthermore, increasing the size of certain components of the blood pump can achieve greater flow rates at lower rotational speeds of the impeller, reducing the likelihood of hemolysis. However, such configurations would take up more space within the elongate shaft 114 of the introducer sheath 100, reducing the space available for the guidewire to pass through. A small diameter guidewire has the advantage of providing smooth and easy tracking through the impeller housing 60 and through the introducer sheath 100, but may not provide enough rail support when navigating through tortuous vascular anatomy.

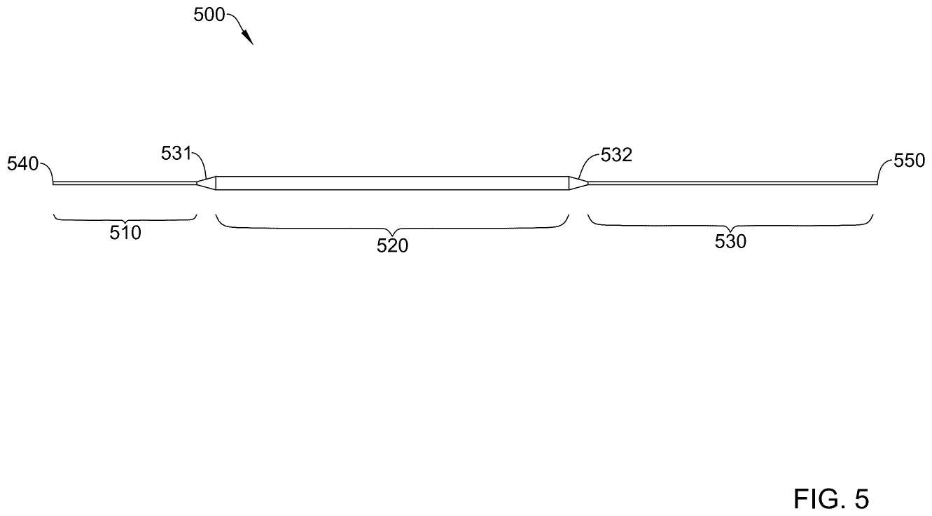

FIG. 5 illustrates an example guidewire 500, which may be representative of the guidewire 46 described above, for use in delivering the blood pump 50, another left ventricular assist device (LVAD), or other medical device to a patient. The guidewire 500 has a distal section 510 extending to a distal end 540 of the guidewire 500, a proximal section 530 extending to a proximal end 550 of the guidewire 500, and a central section 520 extending between the proximal section 530 and the distal section 510. The central section 520 of the guidewire 500 has a large outer diameter, while each of the proximal and distal sections 530, 510 of the guidewire 500 have smaller outer diameters. In some embodiments, the guidewire 500 has transition sections 531, 532 located at the junctions of the central section 537 to the proximal and distal sections 530, 510, respectively. In other words, the guidewire 500 may have a first transition section 531 at the junction between the distal section 510 and the central section 520, and the guidewire 500 may have a second transition section 532 at the junction between the central section 520 and the proximal section 530. The transition sections 531, 532 may be tapered to form a smooth transition between the larger outer diameter of the central section 520 and the smaller outer diameters of each of the proximal and distal sections 530, 510. This may result in the guidewire 500 including three substantially constant diameter elongate sections (e.g., each of the distal, central and proximal sections) and two tapered sections interposed therebetween. In some embodiments, the central section 520 of the guidewire 500 may have an outer diameter of 0.025 inches to 0.035 inches (0.635 mm to 0.90 mm). The proximal section 530 of the guidewire 500 may have an outer diameter less than the outer diameter of the central section 520 and/or the distal section 510 may have an outer diameter less than the outer diameter of the central section 520. In some instances, both the proximal section 530 of the guidewire 500 and the distal section 510 of the guidewire 500 may have an outer diameter less than the outer diameter of the central section 520. In some instances, the proximal section 530 of the guidewire 500 may have an outer diameter between 0.018 inches and 0.035 inches (0.45 millimeters (mm) to 0.90 mm), or between 0.018 inches and 0.025 inches (0.45 mm to 0.635 mm) and/or the distal section 510 of the guidewire 500 may have an outer diameter between 0.018 inches and 0.035 inches (0.45 mm to 0.90 mm), or between 0.018 inches and 0.025 inches (0.45 mm to 0.635 mm). In some embodiments, the proximal section 530 and the distal section 510 of the guidewire 500 may have the same diameter, and in some embodiments, the proximal section 530 and the distal section 510 may have differing diameters.

The guidewire 500 may be of any length required to reach the desired treatment area within a patient. For example, the guidewire 500 may have an overall length between 180 centimeters (cm) (70.87 inches) and 250 cm (98.43 inches), in some instances. The proximal section 530 of the guidewire 500 may have a length between 140 cm (55.12 inches) and 210 cm (82.68 inches). In some embodiments, the length of the proximal section 510 of the guidewire is configured to be longer than the introducer sheath 100 (FIG. 3). For example, the proximal section 530 may be between 50 mm (1.97 inches) and 100 mm (3.94 inches) longer than the introducer sheath in some instances. Thus, once the distal end 540 of the guidewire 500 is positioned at a desired location within the patient's body, such as within the left ventricle of the heart of the patient, the proximal section 530 may extend through the introducer sheath 100, with the second transition section 532, and thus the central section 520 located distal of the distal end of the elongate shaft 114 of the introducer sheath 100. Thus, when the blood pump 50 is advanced through the elongate shaft 114 of the introducer sheath 100, it is being tracked over the proximal section 530 of the guidewire 500 having a smaller outer diameter, and when the blood pump 50 is advanced through the vasculature distal of the introducer sheath 100 it is tracked over the central section 520 having a larger diameter and the distal section 510.

The central section 520 may have any desired length. In some embodiments, the length of the central section 520 is between 300 mm (11.81 inches) and 330 mm (12.99 inches). In some embodiments, the proximal end of the central section 520, and thus the second transition section 532, may be located about 50 mm (1.97 inches) to about 100 mm (3.94 inches) distal of the distal end of the elongate shaft 114 of the introducer sheath 100 while the blood pump 50 is being advanced through the elongate shaft 114 of the introducer sheath 100 over the guidewire 500.

The distal section 510 of the guidewire 500 may have any desired length, for example, between about 30 mm (1.18 inches) and about 70 mm (2.76 inches). The distal section 510 may provide the guidewire 500 with sufficient flexibility to navigate through the vasculature to position the distal section 510 within the ventricle of the heart of the patient, while the central section 520 of the guidewire 500 is positioned in the aorta and extends proximally therefrom toward the vascular access site.

The guidewire 500 may be formed by a variety of methods, including for example, centerless grinding, stamping or rolling, or by other known shape forming methods. In some embodiments the guidewire 500 may include a coating (not shown) that facilitates smooth tracking within a patient's vasculature and reduces friction as the blood pump 50 moves over the guidewire 500. Some coatings may include polytetrafluoroethylene (PTFE), other polymers, ceramics, hydrophilic coatings, or the like. The smaller diameter of the proximal section 530 may also reduce the likelihood of the coating being stripped off of the guidewire 500 as the guidewire 500 passes out of one of the outflow windows 92 of the pump 50.

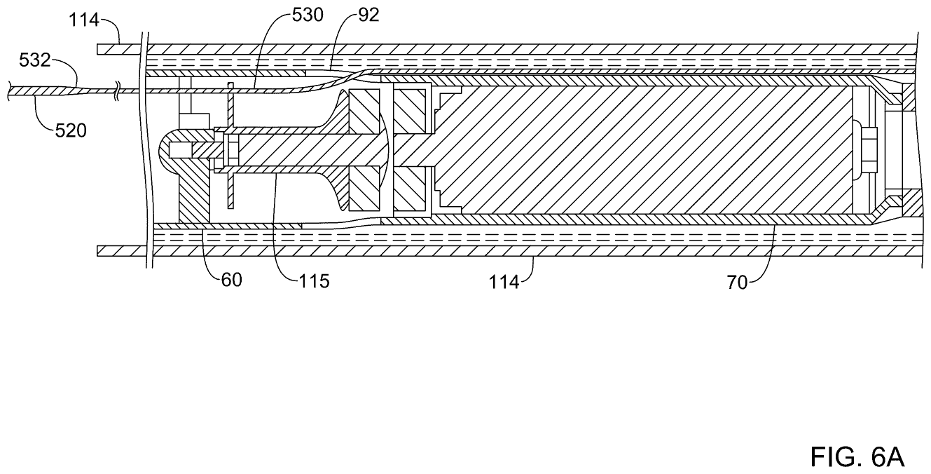

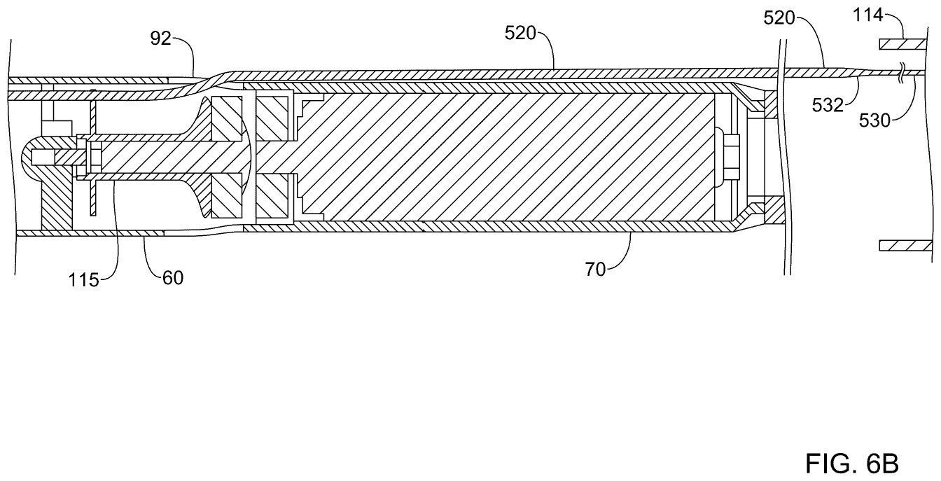

FIG. 6A illustrates a cross-sectional view of the blood pump 50 as it is advanced through the elongate member 114 of the introducer sheath 100 positioned in the vasculature of a patient. As noted above, the guidewire 500 may be advanced through the vasculature to position the distal end of the guidewire at a desired location, such as the left ventricle of the heart of the patient prior to inserting the blood pump 50 through the introducer sheath 100 along the guidewire 500. With the guidewire properly positioned, the smaller diameter proximal section 530 of the guidewire 500 may be arranged within the lumen of the elongate shaft 114 of the introducer sheath 100, with a distal portion of the proximal section 530 extending distal of the distal end of the elongate shaft 114 of the introducer sheath 100. Thus, the second transition region 532, as well as the central section 520 of the guidewire 500 may be located distal of the distal end of the elongate shaft 114 of the introducer sheath 100. The relatively small diameter of the proximal section 530 of the guidewire provides for additional clearance within the elongate member 114 of the introducer sheath 100. As a result, the smaller diameter of the proximal section 530 permits the blood pump 50, including the motor housing 70 and impeller housing 60 to move through the introducer sheath 100 while also permitting the blood pump 50 to have a sufficiently large size. A larger blood pump and/or components of the blood pump 50 may allow for a rigid length of the blood pump 50 to be shorter, which can facilitate easier navigation of the patient's vasculature. The proximal section 530 of the guidewire 500 extends longitudinally through the impeller housing 60, past the impeller 115, and extends out of the outflow window 92. Proximal of the outflow windows 92, the guidewire 530 passes between the exterior of the motor housing 70 and the inner surface of the elongate member 114 of the introducer sheath 100 defining the lumen of the elongate shaft 114. As the blood pump 50 exits the distal end of the elongate shaft 114 of the introducer sheath 100, the blood pump 50 will pass distally beyond the proximal section 530 and the transition region 532, and begin tracking over the larger diameter central section 520 of the guidewire 500.

FIG. 6B illustrates a cross-sectional view of the blood pump 50 as it is tracked along the central section 520 of the guidewire 500 distal of the distal end of the elongate shaft 114 of the introducer sheath 100. Once the guidewire 500 has been advanced into place, the central section 520 is configured to begin distally of the introducer sheath 100, and to facilitate advancement of the blood pump 50 through the vasculature of a patient to a target site, such as across the aortic valve and into the left ventricle. The relatively large diameter of the central section 520 of the guidewire provides increased rail support and control when advancing the blood pump 50 through tortuous anatomy. Though not shown, the blood pump 50 can be advanced distally past the central section 520 and over the smaller diameter distal section 510. The central section of the guidewire 520 extends longitudinally through the impeller housing 60, past the impeller 115, and extends out of the outflow window 92. Proximal of the outflow windows 92, the guidewire 520 extends proximally past the exterior of the motor housing 70.

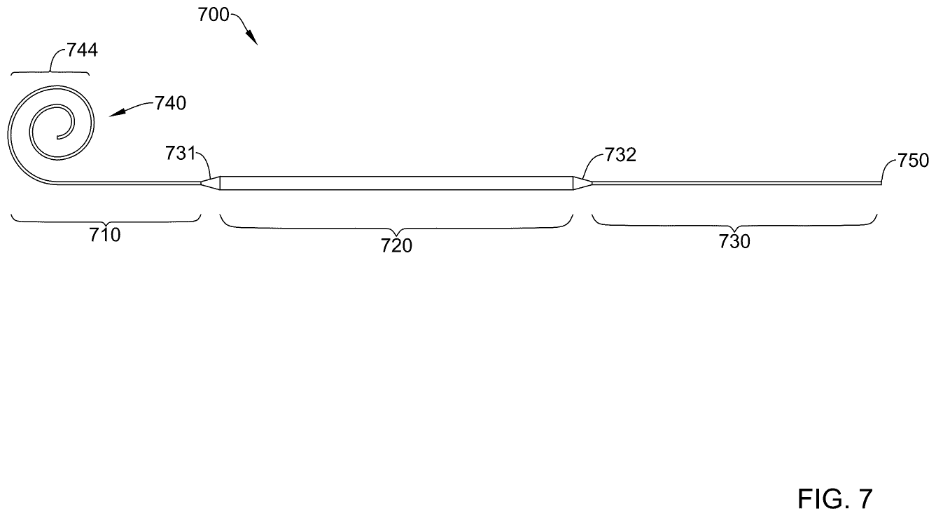

FIG. 7 illustrates another embodiment of a guidewire 700 according to the present disclosure. The guidewire 700 may be representative of the guidewire 46 described above. The guidewire 700 is similar to the guidewire 500 in many respects, and thus the discussion above regarding the guidewire 500 is equally applicable to the guidewire 700, and thus need not be reiterated. The guidewire 700 has a distal section 710 extending to a distal end of the guidewire 700, a proximal section 730 extending to a proximal end 750 of the guidewire 700, and a central section 720 extending between the proximal section 730 and the distal section 710. The central section 720 of the guidewire 700 has a large outer diameter, while each of the proximal and distal sections 730, 710 of the guidewire 700 have smaller outer diameters. In some embodiments, the guidewire 700 has transition sections 731, 732 located at the junctions of the central section 720 to the distal and proximal sections 710, 730, respectively. The guidewire 700 has a curved distal tip. More specifically, a section of the distal section 710 of the guidewire 700 has a preformed recurve section 744.

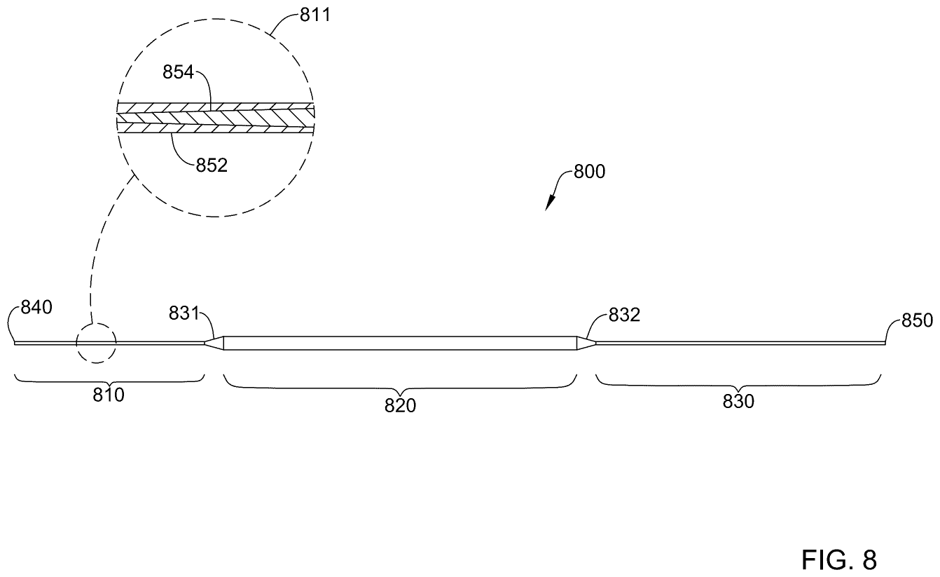

FIG. 8 illustrates another embodiment of a guidewire 800 according to the present disclosure. The guidewire 800 may be representative of the guidewire 46 described above. The guidewire 800 is similar to the guidewire 500 in many respects, and thus the discussion above regarding the guidewire 500 is equally applicable to the guidewire 800, and thus need not be reiterated. The guidewire 800 has a distal section 810 extending to a distal end 840 of the guidewire 800, a proximal section 830 extending to a proximal end 850 of the guidewire 800, and a central section 820 extending between the proximal section 830 and the distal section 810. The central section 820 of the guidewire 800 has a large outer diameter, while each of the proximal and distal sections 830, 810 of the guidewire 800 have smaller outer diameters. In some embodiments, the guidewire 800 has transition sections 831, 832 located at the junctions of the central section 820 to the distal and proximal sections 810, 830, respectively. The guidewire 800 has a flexible distal tip, providing a length of increased flexibility at the distal end of the guidewire 800. In some embodiments, the entire distal section 810 is flexible, thus having a lower bending stiffness or flexural modulus than the center section 820. As shown in cross-sectional view 811, the guidewire 800 may include of a core member 854 and an outer coating 852 surrounding the core member 854. The core member 854 may have a tapered region extending along at least a portion of the length of the distal section 810, which decreases in diameter towards the distal end 840 of the guidewire 800. Though the diameter of the core member 854 decreases in a distal direction, the outer diameter of the guidewire 800 through this length of the distal section 810 may remain constant along the entire length of the distal section 810 in some instances. In some embodiments, the outer diameter of the guidewire 800 may taper towards the distal end 840. The flexible distal tip may have a length between 30 mm (1.18 inches) and 70 mm (2.76 inches), in some instances.

It will be understood that the dimensions described in association with the above figures are illustrative only, and that other dimensions of guidewires, introducer sheaths, and blood pumps are contemplated. The materials that can be used for the various components of the catheter 10, introducer sheath 100, guidewire 500, and the various elements thereof disclosed herein may include those commonly associated with medical devices. For simplicity purposes, the following discussion makes reference to the catheter 10 (and variations, systems or components disclosed herein). However, this is not intended to limit the devices and methods described herein, as the discussion may be applied to other elements, members, components, or devices disclosed herein.

In at least some embodiments, sections, or all of the catheter 10, the introducer sheath 100, and/or the guidewire 500 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the catheter 10, introducer sheath 100, and/or the guidewire 500 (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the catheter 10, the introducer sheath 100, and/or the guidewire 500 (and variations, systems or components thereof disclosed herein) to achieve the same result.

In some embodiments, the catheter 10, the introducer sheath 100, and/or the guidewire 500 (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A percutaneous circulatory support system, comprising:
a guidewire comprising:
a distal section having a first diameter;
a central section having a second diameter; and
a proximal section having a third diameter, the second diameter being greater than both the first diameter and the third diameter;
a blood pump configured to be coupled to and moved along the guidewire, the blood pump comprising:
an impeller housing;
a motor housing coupled to the impeller housing; and
an impeller disposed within the impeller housing, the impeller configured to cause blood to flow through the blood pump.

2. The percutaneous circulatory support system of claim 1, wherein the first and third diameters are equal.

3. The percutaneous circulatory support system of claim 1, wherein the first diameter is less than the third diameter.

4. The percutaneous circulatory support system of claim 1, wherein the guidewire further comprises a first transition section between the proximal section and the central section, and a second transition section between the central section and distal section.

5. The percutaneous circulatory support system of claim 1, further comprising an introducer sheath configured to receive the blood pump and the proximal section of the guidewire therethrough.

6. The percutaneous circulatory support system of claim 1, wherein the impeller housing comprises an inlet and an outlet, wherein the guidewire is configured to extend longitudinally through the impeller housing, proximally out of the outlet, and distally out of the inlet.

7. The percutaneous circulatory support system of claim 6, wherein the proximal section of the guidewire has a length greater than a length of the introducer sheath.

8. The percutaneous circulatory support system of claim 1, wherein the proximal section of the guidewire has a length between 140 cm (55.12 inches) and 210 cm (82.68 inches).

9. The percutaneous circulatory support system of claim 1, wherein the central section of the guidewire has a length between 300 mm (11.82 inches) and 330 mm (12.99 inches).

10. The percutaneous circulatory support system of claim 1, wherein each of the first and third diameters are between 0.457 mm (0.018 inches) and 0.635 mm (0.025 inches), and the second diameter is between 0.635 mm (0.025 inches) and 0.889 mm (0.035 inches).

11. The percutaneous circulatory support system of claim 1, wherein the distal section of the guidewire comprises a tapered core, tapering to a smaller diameter toward a distal end of the guidewire.

12. The percutaneous circulatory support system of claim 11, wherein the guidewire has an overall length of 180 cm (70.87 inches) to 250 cm (98.43 inches).

13. The percutaneous circulatory support system of claim 1, wherein the distal section of the guidewire comprises a preformed recurve portion at a distal end of the guidewire.

14. A percutaneous circulatory support system, comprising:
a guidewire comprising:
a distal section extending to a distal end of the guidewire, the distal section having a first diameter;
a central section having a second diameter;
a proximal section extending to a proximal end of the guidewire, the proximal section having a third diameter, the second diameter being greater than both the first diameter and the third diameter;
a first transition section between the proximal section and the central section; and
a second transition section between the central section and the distal section;
a blood pump configured to be coupled to and moved along the guidewire, the blood pump comprising:
an impeller housing;
a motor housing coupled to the impeller housing; and
an impeller disposed within the impeller housing, the impeller configured to cause blood to flow through the blood pump;
an introducer sheath configured to receive the blood pump and the proximal section of
the guidewire therethrough.

15. The percutaneous circulatory support system of claim 14, wherein the first and third diameters are equal.

16. The percutaneous circulatory support system of claim 15, wherein the first and third diameters are between 0.457 mm (0.018 inches) and 0.635 mm (0.025 inches).

17. The percutaneous circulatory support system of claim 16, wherein the second diameter is between 0.635 mm (0.025 inches) and 0.889 mm (0.035 inches).

18. A method of positioning a blood pump within a patient, the method comprising:
inserting an introducer sheath at an access point;
inserting a guidewire into the introducer sheath, the guidewire having a distal section with a first diameter, a central section with a second diameter, and a proximal section with a third diameter, the first and third diameters being less than the second diameter;
positioning the guidewire through the introducer sheath such that a distal end of the proximal section is distal of the introducer sheath; and
thereafter, advancing the blood pump through the introducer sheath along the proximal section of the guidewire, the blood pump comprising an impeller housing and an impeller disposed within the housing and configured to cause blood to flow through the blood pump.

19. The method of claim 18, wherein the third diameter is 0.635 mm (0.025 inches) or less and the second diameter is greater than 0.635 mm (0.025 inches).

20. The method of claim 18, further comprising advancing the blood pump distally out of the introducer sheath and along the central section of the guidewire.
